(19) United States
(12) Patent Application Publication
(10) Pub. No.: US 2023/0333106 A1
(43) Pub. Date: Oct. 19, 2023

(54) COMPOSITIONS AND METHODS FOR EARLY DETECTION OF OSTEOPOROSIS

(71) Applicant: New York Society for the Relief of the Ruptured and Crippled, maintaining the Hospital for Special, New York, NY (US)

(72) Inventor: Kyung-Hyun Park-Min, Fort Lee, NJ (US)

(21) Appl. No.: 18/042,730

(22) PCT Filed: Aug. 24, 2021
(86) PCT No.: PCT/US2021/047283

§ 371 Date: Feb. 23, 2023

Related U.S. Application Data
(60) Provisional application No. 63/069,477, filed on Aug. 24, 2020.

(51) Int. Cl. G01N 33/569

(52) U.S. Cl. CPC G01N 33/56972; G01N 2800/108; G01N 2333/705; G01N 2800/52; G01N 2800/56

(57) ABSTRACT

Provided herein are methods of diagnosing and treating a skeletal disease in a subject. The method includes (i) identifying the presence of osteoclast-enhancing cells (OECs) in the subject; (ii) quantifying the number of circulating OECs in a sample from the subject; and (iii) diagnosing a skeletal disease in the subject when an increase in OECs is detected as compared to a control.

### STATEMENT REGARDING FEDERALLY SPONSORED RESEARCH OR DEVELOPMENT

This invention was made with government support under UL1 TR002384 awarded by the National Institutes of Health. The government has certain rights in the invention.

### BACKGROUND

Osteoporosis is a worldwide disease with reduction of bone mass and decrease of bone strength to result in bone fragility and fracture. It is predicted that more than 200 million women aged 50 and older are affected by osteoporosis worldwide, and 54 million Americans with osteoporosis and low bone mass have been reported. Every year, osteoporosis is responsible for 2 million cases of bone fractures. Given the aging population, by 2025, annual direct costs from osteoporosis are expected to reach approximately $25.3 billion (National Osteoporosis Foundation. Fast Facts. http://www.nof.org/node/40. Accessed on Jul. 25, 2020) (J. J. Roche, R. T. Wenn, O. Sahota, C. G. Moran, Effect of comorbidities and postoperative complications on mortality after hip fracture in elderly people: prospective observational cohort study. BMJ 331, 1374 (2005), incorporated herein by reference). Thus, osteoporosis has significant physical, emotional, and financial consequences.

Osteoporosis is a silent disease without obvious symptoms or evidence until occurrence of fracture. Early diagnosis of osteoporosis is the key issue for efficient treatment and for identification of osteoporotic patients with high risk of fracture. The diagnosis of osteoporosis and assessment of fracture risk are based on the quantitative analysis of bone mineral density (BMD) by dual-energy x-ray absorptiometry (DXA) (G. M. Blake, I. Fogelman, The role of DXA bone density scans in the diagnosis and treatment of osteoporosis. Postgrad Med J 83, 509-517 (2007), incorporated herein by reference). However, the gold standard method of BMD assessment of bone mass by DXA only partially provides information about bone strength. In addition, changes in radiographically detectable bone mass may be delayed from several months to more than a year for specific insults or treatments that affect bone mass. Therefore, a readout that responds more rapidly to changes in bone physiology is desired (C. H. Chesnut, 3rd, McClung, M. R., Ensrud, K. E., Bell, N. H., Genant, H. K., Harris, S. T., Singer, F. R., Stock, J. L., Yood, R. A., and Delmas, P. D, Alendronate treatment of the postmenopausal osteoporotic woman effect of multiple dosages on bone mass and bone remodeling. Am J Med 99, 144-152 (1995), incorporated herein by reference).

Several assays are available for measuring bone turnover markers (BTMs). These assays measure collagen breakdown products and other molecules released from osteoclasts and osteoblasts during the process of bone resorption and formation. The use of BTMs in clinical trials has helped to understand the mechanism of action of osteoporosis drugs (R. Civitelli, R. Armamento-Villareal, N. Napoli, Bone turnover markers: understanding their value in clinical trials and clinical practice. Osteoporos Int 20, 843-851 (2009), incorporated herein by reference). However, the measurement of BTMs is complicated by large, random, within-patient variability; biologic variability (age, gender, body mass index, circadian, and menstrual variation); and poor standardization of most assays (T. T. Hlaing, J. E. Compston, Biochemical markers of bone turnover—uses and limitations. Ann Clin Biochem 51, 189-202 (2014), K. J. Bell et al., The potential value of monitoring bone turnover markers among women on alendronate. J Bone Miner Res 27, 195-201 (2012), incorporated herein by reference). Biologic and laboratory variability in BTMs values has confounded their widespread use in clinical practice.

Therefore, discovering new diagnostic markers that closely correlate with early osteoporosis disease activity is imperative.

### SUMMARY OF THE INVENTION

Provided herein are compositions and methods relating to osteoclast enhancing cells (OECs). As demonstrated herein, depletion of the OEC population from CD14+ monocytes suppressed in vitro osteoclast differentiation. Further, the number of circulating OECs is highly increased in individuals who have low bone density including patients with osteoporosis and osteopenia compared to OECs of healthy donors. The number of OECs shows a strong negative correlation with bone density, and the frequency of OECs is decreased in osteoporosis patients with antiresorptive therapy along with increased bone density.

Provided herein, in one aspect is a method of diagnosing and treating a skeletal disease in a subject. The method includes (i) identifying the presence of osteoclast-enhancing cells (OECs) in the subject; (ii) quantifying the number of circulating OECs in a sample from the subject; and (iii) diagnosing a skeletal disease in the subject when an increase in OECs is detected as compared to a control. In one embodiment, the method further includes treating the subject for the skeletal disease, optionally, osteoporosis. In another aspect, a method of diagnosing an increased risk of a skeletal disease in a subject. The method includes (i) identifying the presence of osteoclast-enhancing cells (OECs) in the subject; (ii) quantifying the number of circulating OECs in a sample from the subject; and (iii) diagnosing an increased risk of skeletal disease in the subject when an increase in OECs is detected as compared to a control. In one embodiment, the subject is given a treatment following the determination of increased risk of disease.

In another aspect, a method of assessing the efficacy of a treatment for a skeletal disease is provided. The method includes (i) identifying the presence of osteoclast-enhancing cells (OECs) in the subject; (ii) quantifying the number of circulating OECs in a sample from the subject. A decrease in the number of circulating OECs in the subject, as compared to the start of treatment is indicative of efficacy of treatment. In one embodiment, the treatment is a bisphosphonate.

In another aspect, a method of diagnosing an increased risk of a surgical complication in a subject prior to surgery is provided. The method includes (i) identifying the presence of osteoclast-enhancing cells (OECs) in the subject; (ii) quantifying the number of circulating OECs in a sample from the subject; and (iii) diagnosing an increased risk of a surgical complication in the subject when an increase in OECs is detected as compared to a control. In one embodiment, the subject is given a treatment following the determination of increased risk of complication.

Other aspects and advantages of the invention will be readily apparent from the following detailed description of the invention.

### DESCRIPTION OF THE FIGURES

FIG. 1A-1D show that a subset of CD14⁺ monocytes expresses RANK. Human PBMCs were isolated from healthy donors. Human PBMCs were stained and analyzed with flow cytometry. A. UMAP plot showing the different cell populations. B. UMAP plot, color coding (red to blue) for the expression of CD14, a marker gene of human monocytes. C. UMAP plot of CD14 positive cells for the expression of RANK. D. Cumulative data showing the histograms of CCR2, C3AR1, CD66b, CD51/CD61, HLA-DR, and CD14 expression levels of CD14⁺RANK⁺ cells from three independent donors.

FIG. 2A-FIG. 2C show that osteoclast-enhancing cells (OECs) enhance osteoclastogenesis. A. Representative flow plots for human PBMCs. Among the CD14+ population, RANK high/CD66b negative cells are named as osteoclast-enhancing cells (OECs) and RANK negative/CD66b negative cells are named as monocytes (MOs). B-C. Representative histograms of CCR2, C3AR1, CD66b, CD51/CD61, HLA-DR, and CD14 expression levels of OECs.

FIG. 3A-3H shows that OECs increase in patients with osteoporosis. A. Immunohistochemistry of DAPI and NFATc1. CD14+ cells from patient with osteoporosis and healthy donors with normal BMD are cultured with M-CSF and RANKL for one day. Then the cells are stained with anti-NFATc1 antibodies. Left panels show representative images of DAPI (nucleus stain) and NFATc1. Scale bar: 100 μm. The right panel shows the percentages of NFATc1 positive cells per total cells. B-H. OECs from healthy donors (n=9), osteopenia patients (n=19), or osteoporosis patients (n=16) who are diagnosed by the T-score of a DXA test are counted by flow cytometry. Bone density is determined by a DXA test. B. Representative flow plots showing RANK and CD66b expression of CD14+ cells. C. A plot shows the absolute count of CD14+ cells per μl of blood. D. A plot shows the absolute count of OECs per μl of blood. E. A correlation plot between the absolute count of OECs per μl of blood and lumbar BMD (n=44). OECs are counted by FACS analysis and lumbar BMD is measured by DXA analysis. F. A correlation plot between CTX and the number of OECs. G. A correlation plot between ALP and the number of OECs. H. A correlation plot between P1NP and the number of OECs. All data are shown as mean±SEM. *P<0.05; **P<0.01; ns, not significant by two-tailed, unpaired t-test in A; one-way ANOVA with a post hoc Tukey test in C and D, or Spearman correlation test in E, F, G, and H.

FIG. 4A-4D shows that OECs have a distinct transcriptomic signature. A. Volcano plot of RNA-seq analysis of differentially expressed genes (DEGs) in OECs and MOs. Significantly regulated genes (FDR<0.01 and 1.5 fold changes) are marked as red. B. Top 10 canonical pathways by Ingenuity Pathway Analysis of differentially expressed genes. C. Heatmap showing relative expression (z-score) of 926 genes differentially expressed with FDR<0.01 in OECs versus MOs from 2 replicates. One sample contains cumulative data from 6 donors and the other sample has cumulative data from 5 donors. D. Upstream regulator analysis of significantly regulated DEGs.

FIG. 5A-5C demonstrates an osteoclastogenesis assay in CD14+ cells with or without OECs. OECs and CD14+ cells without OECs are sorted by FACS analysis. FIG. 5A shows representative images of TRAP-stained cells. FIG. 5B shows the percentage of TRAP-positive multinuclear cells (MNCs: more than three nuclei) per control. FIG. 5C shows Giemsa-stained monocytes (MOs) and osteoclast enhancing cells (OECs) that are sorted by FACS. Scale bar: 100 μm. All data are shown as mean ±SEM. *P<0.05; **P<0.01; ns, not significant by two-tailed, unpaired t-test in C.

FIG. 6 is a plot showing the number of OECs in patients with osteoporosis with or without bisphosphonate treatment. Bisphosphonate (n=11) and No treatment group (n=16).

FIGS. 7A and 7B show the frequency of OECs in subjects with normal BMD or with osteoporosis.

FIG. 8 demonstrates that measuring the frequency of OECs may be an alternative assessing tool for osteoporosis treatment. FIG. 8 is a graph showing T-score (bone mineral density) vs. circulating OECs of 11 osteoporosis patients treated with bisphosphonate.

### DETAILED DESCRIPTION OF THE INVENTION

Osteoporosis is a metabolic bone disorder characterized by compromised bone mass, quality, and strength, resulting in an increased risk of fracture. More than 25% of the middle-age population has low bone mass and most remain unaware of their growing risk for fracture. Although considerable work has been done to improve diagnostic standards and tools to identify low bone mass, early detection of the predicate causes of osteoporosis does not exist. Therefore, discovering new diagnostic markers that closely correlate with early osteoporosis disease activity is imperative. Described herein is a new cellular marker that closely correlates with increased osteoclast activity, a prime underlying cause responsible for pathological bone loss. Compositions and methods which incorporate this cellular marker, termed osteoclast enhancing cells (OEC), are provided.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human The subject may be of any age, as determined by the health care provider. In certain embodiments described herein, the patient is a subject who has or is at risk of developing a skeletal disease. The subject may have been treated for a skeletal disease previously, or is currently being treated for the skeletal disease. In another embodiment, the patient is a subject who has undergone, or will undergo within the next month, orthopedic surgery. In one embodiment, the subject is a female. In one embodiment, the subject is a pre-menopausal woman In another embodiment, the subject is a post-menopausal woman. In one embodiment, the subject is an older adult, e.g., over the age of 40. In another embodiment, the subject is at least 45, 50, 55, or 60 years of age. In yet another embodiment, the subject is a senior adult, i.e., over 60 years of age. In another embodiment, the subject has been diagnosed with, or is suspected of having, cancer. In another embodiment, the subject has been diagnosed with, or is suspected of having, osteoporosis. In another embodiment, the subject has been diagnosed with, or is suspected of having, osteopenia.

As used herein, the term “skeletal disease” or “skeletal disorder” refers to any condition associated with the bone or joints, including those associated with bone loss, bone fragility, or softening, or aberrant skeletal growth. Skeletal diseases include, without limitation, osteoporosis and osteopenia, rheumatoid arthritis, osteoarthritis, psoriatic arthritis, periodontitis, periprosthetic loosening, osteomalacia, hyperparathyroidism, Paget disease of bone, spondyloarthritis, and lupus. In one embodiment, the skeletal disease is osteoporosis. In one embodiment, the skeletal disease is osteopenia. In another embodiment, the skeletal disease or disorder is osteolytic bone loss resulting from cancer metastasis. In yet another embodiment, the skeletal disease or disorder is rheumatoid arthritis.

“Sample” as used herein means any biological fluid or tissue that contains cells or tissue, including blood cells, fibroblasts, and skeletal muscle. In one embodiment, the sample is whole blood. In another embodiment, the sample is peripheral blood mononuclear cells (PBMC). Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

As used herein, the term “a therapeutically effective amount” refers an amount sufficient to achieve the intended purpose. For example, an effective amount of a therapy for skeletal disease is sufficient to decrease osteoclastogenesis or osteoclast function, bone resorption or destruction in a subject. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

As used herein, “disease”, “disorder” and “condition” are used interchangeably, to indicate an abnormal state in a subject.

Osteoclast-Enhancing Cells

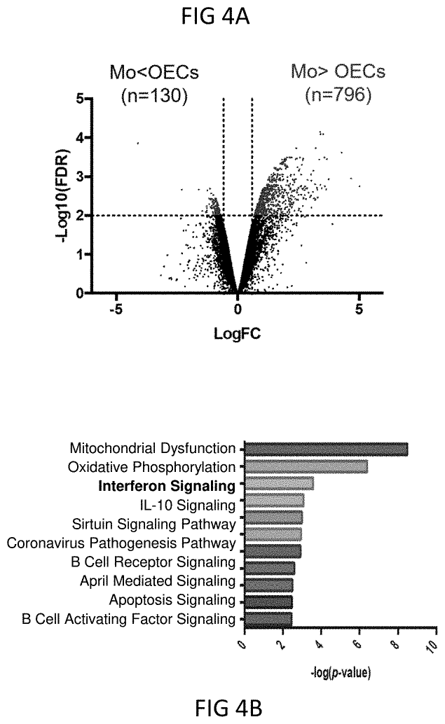

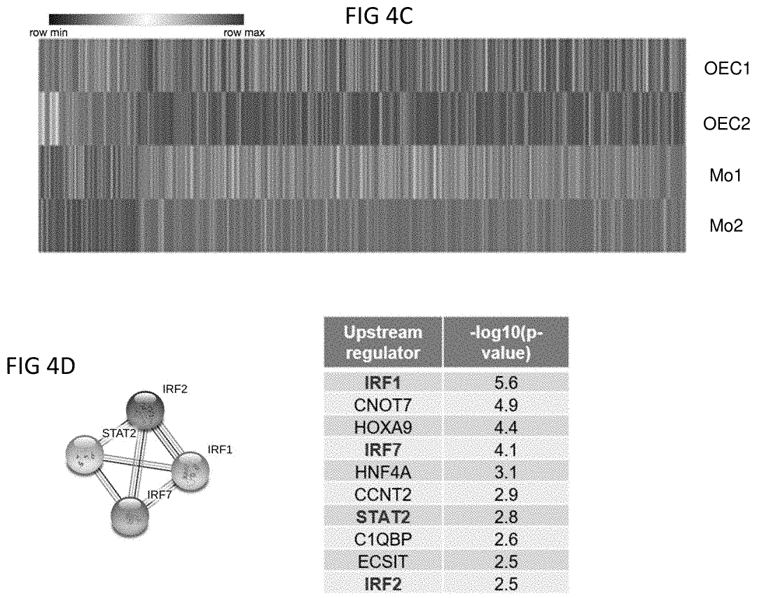

Described herein is a new circulating cell population from human blood, termed osteoclast-enhancing cells (OECs). OECs enhance human osteoclastogenesis and express essential osteoclast-specific receptors such as CSF1R and RANK, but do not express CD66b, CD3, and CD19. OECs also express CD14, but the morphology of OECs differs from CD14+monocytes. Further, RNA-sequencing analysis shows that the expression of 2697 genes is significantly (p<0.05, Fold Changes>1.5) changed between OECs and monocytes (FIG. 4C). Ingenuity Pathway Analysis identified that the differentially expressed genes are enriched in key regulatory pathways for osteoclastogenesis including oxidative stress and interferon signaling pathways.

Osteoclastogenesis is the formation of bone-resorbing cells, called osteoclasts, from precursor cells of myeloid origin. A physical contact of precursor cells with osteoblasts or other specific mesenchymal cells, such as stromal or synovial cells, is essential for osteoclastogenesis. Osteoclasts are the exclusive cell type responsible for bone resorption in both bone homeostasis and pathological bone destruction. As provided herein, the number of circulating OECs provides a marker for increased osteoclastic activity in bone. As shown in the examples, the frequency of OECs in blood from postmenopausal women with no treatment was determined. The number of OECs was then compared with lumbar spine and total hip bone mineral density (BMD) from DXA and serum bone turnover markers. Of the 53 participants, 31 women (58%) had osteoporosis (mean age±SD: 67.5±10.0), 23 (43%) or osteopenia (66.3±8.3), and 9 (17%) had normal BMD (66.1±9.1). Correlation analysis showed a significant correlation between the number of OECs and BMD (p<0.0011, r=−0.4823), and between the number of OECs and CTX (p=0.0047, r=0.5275), a known osteoporosis marker. Therefore, a circulating population of OECs with high osteoclastogenic activity is described and significantly increased OECs have been found in subjects with the lowest BMD values. These data show that OECs serves as a prognostic marker for patients with low and falling bone mass.

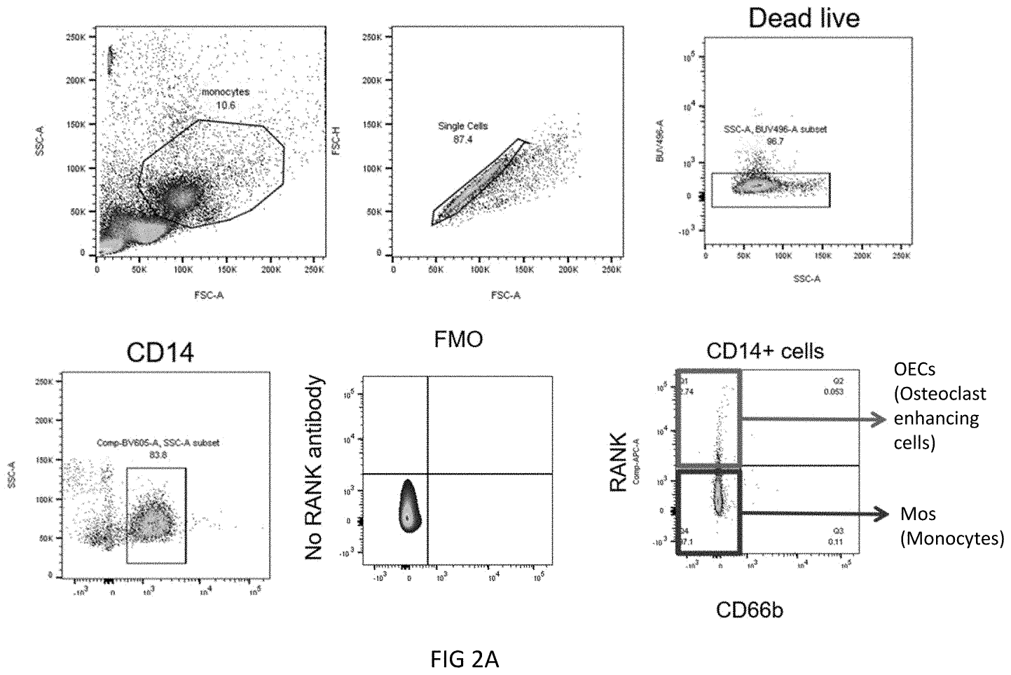

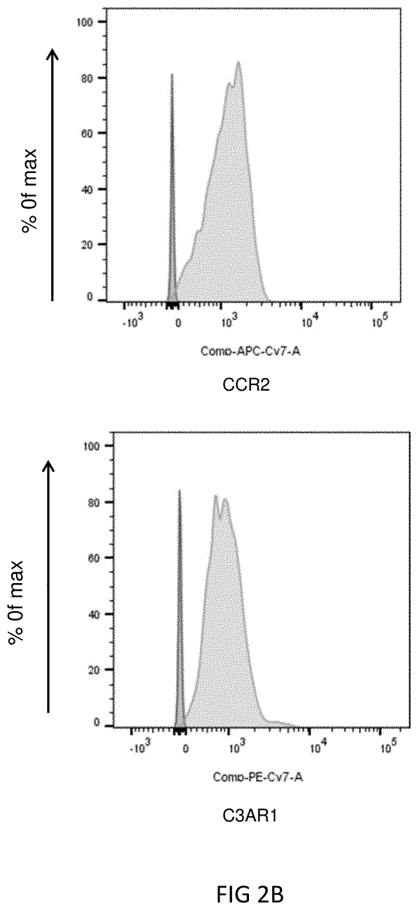

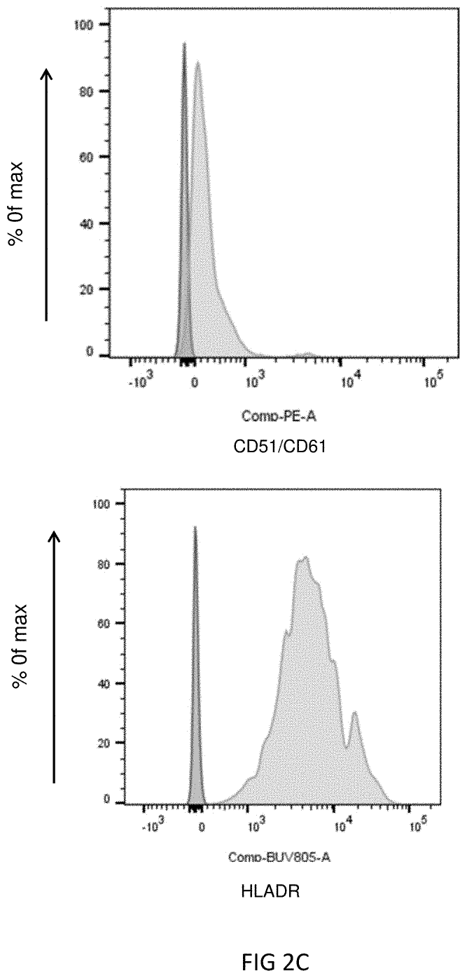

In one aspect, an isolated population of cells comprising osteoclast enhancing cells (OECs) is provided. As discussed herein, osteoclast enhancing cells are a newly characterized CD14⁺ cell type, that differs from CD14⁺ monocytes by morphology. See, FIG. 2C and 2D. Further, OECs have the phenotype CD14⁺RANK⁺CD66b⁻CCR2⁺C3AR⁺CD51/61^{low}HLADR⁺. A population of OECs may be enriched from a starting sample of peripheral blood mononuclear cells (PBMC), using known cell sorting techniques, such as FACS, and one or more of these cell surface markers. For example, antibodies such as those shown in Table 1 are useful in enriching the OEC population.

Methods of sorting cells, for example, by FACS are known in the art. Briefly, PBMCs are incubated with saturating concentrations of the monoclonal antibodies of Table 1 at 4° C. for 25 min. Subsequently, red cells are lysed with ACK Lysing Buffer (Gibco Life Technologies, Grand Island, NY), washed twice, resuspended in PBS containing 1% BSA and analyzed on a FACSCanto II (BD Life Sciences, Franklin Lakes, NJ) or FACS Symphony (BD Life Sciences, Franklin Lakes, NJ). For cell sorting, stained cells are fractionated by using a BD Influx Cell Sorter (BD Life Sciences, Franklin Lakes, NJ). In one embodiment, the OECs are further diluted with saline, buffer or a physiologically acceptable diluent. Alternatively, such cell populations are concentrated by conventional means.

In another aspect, the OECs described herein, are utilized in clinical methods to enable diagnosis, evaluation and/or treatment of subjects in need thereof. In one aspect, a method of diagnosing an increased risk of developing a skeletal disease is provided. The method includes one or more of: identifying the presence of OECs in a sample from a subject and quantifying the number of circulating OECs in the sample. In one embodiment, the sample is whole blood. In another embodiment, the sample is PBMCs. In some embodiments, the presence or amount of OECs is detected in a sample obtained from a subject. This level may be compared to the level of a control. In one embodiment, detection of, or an increase in the number of OECs, as compared to a control indicates a risk of developing a skeletal disease. As used herein an “increased risk of skeletal disease” means a greater risk as compared to a control. In one embodiment, as used herein, the term “control” refers to an average subject having at least some common demographics as the subject. For example, if the subject is a menopausal woman, the risk of developing a skeletal disease may be compared to the risk of an average menopausal woman of the same age in developing the skeletal disease. “Control” or “control level” as used herein refers to the source of the reference value for OEC levels, or risk level. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom. In one embodiment, the control is a subject having a level of under 20 OEC cells/uL. In one embodiment, the control is a subject having normal bone density.

In one embodiment, a level of under 20 cells/uL is indicative of a low risk of developing a skeletal disease, as compared to a control. In another embodiment, a level of about 21 cells/uL to about 80 cells/uL is indicative of a medium risk of developing a skeletal disease, as compared to a control. In another embodiment, a level of over 80 cells/uL is indicative of a high risk of developing a skeletal disease in the subject, as compared to a control. In one embodiment, the skeletal disease is osteoporosis. In another embodiment, the skeletal disease is fracture.

In one embodiment, a level of under 20 cells/uL indicates that the subject has a normal bone density, i.e., does not have osteoporosis or osteopenia. In another embodiment, a level of about 21 cells/uL to about 80 cells/uL indicates that the subject has osteopenia. In another embodiment, a level of about 30 cells/uL to about 75 cells/uL indicates that the subject has osteopenia. In another embodiment, a level of over 80 cells/uL indicates that the subject has osteoporosis.

The presence or level of OECs may be determined by the person of skill in the art, e.g., by the use of FACS. In one embodiment, the cells are detected by the identification of cell surface markers. In one embodiment, the OECs have the phenotype CD14+/RANK+/CD66−. In another embodiment, the OECs have the phenotype CD14+RANK+CD66b−CCR2+C3AR+CD51/61+HLADR+. Antibodies useful in detecting the presence or level of OECs are known in the art and include, e.g., those shown in Table 1. In another embodiment, the number of OECs in the sample are determined using an automated cell counting machine. In another embodiment, the number of OECs in the sample are determined using microscopy.

In another aspect, a method of diagnosing a skeletal disease in a subject is provided. The method includes one or more of: identifying the presence of OECs in a sample from a subject and quantifying the number of circulating OECs in the sample. In one embodiment, the sample is whole blood. In another embodiment, the sample is PBMC. In some embodiments, the presence or amount of OECs is detected in a sample obtained from a subject. In one embodiment, the level of OECs is compared to a control level. In one embodiment, detection of, or an increase in the number of OECs, as compared to a control indicates the presence of a skeletal disease. In one embodiment, the skeletal disease is osteoporosis. In one embodiment, a level of 80 cells/uL or higher is indicative of the presence of a skeletal disease in the subject. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom. In one embodiment, the method further includes treating the subject for the skeletal disease.

In one embodiment, the method of diagnosing a skeletal disease, or an increased risk of skeletal disease, includes treatment with an appropriate therapeutic. Such therapies include without limitation, nonsteroidal anti-inflammatory drugs (NSAIDs), steroids such as prednisone, methotrexate (Trexall, Otrexup, others), leflunomide (Arava), hydroxychloroquine (Plaquenil) and sulfasalazine (Azulfidine), abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Other additional therapies include Bisphosphonates including Alendronate (Fosamax), Risedronate (Actonel), Ibandronate (Boniva), and Zoledronic acid (Reclast). Other therapies include hormone like medications including raloxifene (Evista), Denosumab (Prolia, Xgeva), Teriparatide (Forteo), Abaloparatide (Tymlos).

In one embodiment, the method includes quantifying the number of circulating OECs in a sample from a subject. In one embodiment, the sample is whole blood. In another embodiment, the sample is PBMC. In one embodiment, detection of, or an increase in the number of OECs, as compared to a control indicates the presence of a skeletal disease. The method includes treating the subject with a bisphosphonate.

Currently, a number of effective treatments for osteoporosis are available, which increase bone mineral density (BMD) and decrease the risk of fractures. Although monitoring changes in BMD or bone turnover markers are used to assess response to treatment, there is no clear consensus on the optimal method for assessing response to treatment in an individual patient. In addition, these changes may be misleading due to the threshold for the magnitude of the changes. Therefore, it is hard to quantify the patients who fail to respond to osteoporosis therapy. It has been shown that about 15% of patients on bisphosphonate therapy fail to respond to bisphosphonate. As shown in Example 4, and FIG. 8, measuring the frequency of OECs is an alternative tool for assessing osteoporosis treatment.

Thus, in another aspect, a method of determining the suitability of bisphosphonate treatment for a skeletal disease is provided. The method includes one or more of: identifying the presence of OECs in a sample from a subject prior to receiving bisphosphonate treatment, and quantifying the number of circulating OECs in the sample. In one embodiment, the sample is whole blood. In another embodiment, the sample is PBMC. In some embodiments, the presence or amount of OECs is detected in a sample obtained from the subject. In one embodiment, a level of 80 cells/uL or less indicates that the subject will likely respond to bisphosphonate, i.e., is a “responder”. In certain embodiments, a level of 100 cells/uL, 90 cells/uL, 80 cells/uL, 70 cells/uL, 60 cells/uL, 50 cells/uL, 40 cells/uL, 30 cells/uL, 20 cells/uL or less is a responder. In one embodiment, the method further includes treating the subject with bisphosphonate when the test indicates that the subject is a responder.

In another aspect, a method of determining whether a subject is responding to bisphosphonate treatment is provided. The method includes one or more of: identifying the presence of OECs in a sample from a subject who is currently receiving, or has received, bisphosphonate treatment, and quantifying the number of circulating OECs in the sample. In one embodiment, the sample is whole blood. In another embodiment, the sample is PBMC. In some embodiments, the presence or amount of OECs is detected in a sample obtained from the subject. In one embodiment, a level of 80 cells/uL or less indicates that the subject will likely respond to bisphosphonate, i.e., is a “responder”. In certain embodiments, a level of 100 cells/uL, 90 cells/uL, 80 cells/uL, 70 cells/uL, 60 cells/uL, 50 cells/uL, 40 cells/uL, 30 cells/uL, 20 cells/uL or less will be a responder. In one embodiment, the method further includes treating the subject with bisphosphonate when the test indicates that the subject is a responder.

In another embodiment, a method of assessing a patient for risk of complication prior to orthopedic surgery is provided. The method includes quantifying the number of circulating OECs in a sample from a subject. In one embodiment, detection of, or an increase in the number of OECs, as compared to a control indicates an increased risk of complications from surgery. In one embodiment, the method includes performing additional testing on the subject. Such testing may include a DXA scan. In another embodiment, a pharmaceutical, or other treatment is provided if an increased risk of complications from surgery is diagnosed. Such treatment includes a cytotoxic drug conjugated to an antibody that targets OECs. In another embodiment, treatment includes any of those described herein for skeletal disease.

In another aspect, a method of assessing the efficacy of a treatment for a skeletal disease is provided. In one embodiment, a baseline level of OECs is obtained from the subject prior to, or at the beginning of treatment for a skeletal disease. After a desirable time period, the level of OECs in the subject is measured again. A decrease in the level of OECs as compared to the earlier time point indicates that the treatment for the skeletal disease is, at least partially, efficacious. The treatment may be any of those described herein, or other treatments deemed suitable by the health care provider. In one embodiment, the treatment regimen is altered based on the level of OECs detected.

Such treatments include without limitation, nonsteroidal anti-inflammatory drugs (NSAIDs), steroids such as prednisone, methotrexate (Trexall, Otrexup, others), leflunomide (Arava), hydroxychloroquine (Plaquenil) and sulfasalazine (Azulfidine), abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Other treatments include Bisphosphonates including Alendronate (Fosamax), Risedronate (Actonel), Ibandronate (Boniva), and Zoledronic acid (Reclast). Other treatments include hormone like medications including raloxifene (Evista), Denosumab (Prolia, Xgeva), Teriparatide (Forteo), and Abaloparatide (Tymlos). Other treatments include those for metastatic bone loss.

In any of the methods described herein, the subject may have, or be suspected of having or developing, a skeletal disease, as described hereinabove. In one embodiment, the subject has, or is suspected of having or developing, rheumatoid arthritis. In another embodiment, the subject has, or is suspected of having or developing, psoriatic arthritis. In another embodiment, the subject has, or is suspected of having or developing, periodontitis. In another embodiment, the subject has, or is suspected of having or developing, periprosthetic loosening. In another embodiment, the subject has, or is suspected of having or developing, osteoporosis. In another embodiment, the subject has, or is suspected of having or developing, metastatic bone loss.

In one embodiment, a method of diagnosing and treating a skeletal disease in a subject is provided. The method comprises one or more of identifying the presence of osteoclast-enhancing cells (OECs) in the subject; quantifying the number of circulating OECs in a sample from the subject; diagnosing a skeletal disease in the subject when an increase in OECs is detected as compared to a control; and treating the subject for the skeletal disease. In one embodiment, the skeletal disease is osteoporosis or osteopenia. In one embodiment, the OECs are detected by identification of cell surface markers. In one embodiment, the OECs have the phenotype CD14+/RANK+/CD66−. In another embodiment, the OECs have the phenotype CD14+RANK+CD66b−CCR2+C3AR+CD51/61+HLADR+. In one embodiment, the subject is treated for osteoporosis using antiresorptive therapy.

In another embodiment, a method of diagnosing an increased risk of a skeletal disease in a subject is provided. The method comprises one or more of identifying the presence of osteoclast-enhancing cells (OECs) in the subject; quantifying the number of circulating OECs in a sample from the subject; diagnosing an increased risk of skeletal disease in the subject when an increase in OECs is detected as compared to a control. In one embodiment, the method includes treating the subject for increased risk of skeletal disease. In one embodiment, the skeletal disease is osteoporosis or osteopenia. In one embodiment, the OECs are detected by identification of cell surface markers. In one embodiment, the OECs have the phenotype CD14+/RANK+/CD66−. In another embodiment, the OECs have the phenotype CD14+RANK+CD66b−CCR2+C3AR+CD51/61+HLADR+. In one embodiment, the subject is treated for increased risk of osteoporosis using antiresorptive therapy.

In another embodiment, a method of assessing the efficacy of a treatment for a skeletal disease is provided. In one embodiment, the method includes one or more of identifying the presence of osteoclast-enhancing cells (OECs) in the subject, and quantifying the number of circulating OECs in a sample from the subject, and comparing the number of circulating OECs to a control. In one embodiment, a a decrease in the number of circulating OECs as compared to a control indicates the treatment is at least partially efficacious. In one embodiment, the treatment is a bisphosphonate. In another embodiment, the control is an OEC level obtained from the subject at an earlier time point.

In yet another embodiment, a method of diagnosing an increased risk of a surgical complication in a subject prior to surgery is provided. In one embodiment, the method includes one or more of identifying the presence of osteoclast-enhancing cells (OECs) in the subject, and quantifying the number of circulating OECs in a sample from the subject. An increased risk of a surgical complication in the subject is diagnosed when an increase in OECs is detected as compared to a control. In one embodiment, the subject is treated for increased risk of complication.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

The following examples are illustrative only and are not intended to limit the present invention.

### EXAMPLES

Osteoporosis is a metabolic bone disorder that compromises bone strength and leads to an increased risk of fracture. Skeletal fractures caused by osteoporosis lead to morbidity and an increased risk of mortality; such fractures are also associated with expensive care costs. Thus, osteoporosis represents a serious public health problem, and both early diagnosis and effective therapies for osteoporosis are urgently needed. However, current diagnostic methods are not suitable to detect the risk of fracture early, and the available anti-resorptive drugs that are effective in inhibiting bone resorption have significant side effects. As described herein, the inventors have developed early diagnostic biomarkers of osteoporosis or pathological bone loss.

The key findings are as follows: 1. Depletion of the OEC population from CD14+ monocytes suppressed in vitro osteoclast differentiation. 2. The number of circulating OECs was highly increased in individuals who have low bone density including patients with osteoporosis and osteopenia compared to OECs of healthy donors. 3. The number of OECs showed the strong negative correlation with bone density. 4. The frequencies of OECs were decreased in osteoporosis patients with antiresorptive therapy along with increased bone density.

Example 1: Materials and Methods

Subjects

Participants were forty-four (mean age±SD: 67.3±9.2 years) postmenopausal women with osteoporosis, who visited Hospital for special surgery for assessment of osteoporosis. Of the 44 participants, 16 women (36%) had osteoporosis (67.3±8.8), 19 (43%) had osteopenia (66.8±8.8), and 9 (20%) had normal BMD (66.1±9.5). This study was approved by the Hospital for Special Surgery Institutional Review Board (2016-0663). All subjects agreed to participate in this study and gave informed consent.

Measurement of Bone Mineral Density (BMD)

Hip (total hip regions) and lumbar spine (L2-L4 region) BMD was evaluated by using DXA on a Horizon A (Hologic, Waltham, MA). Information from the DXA scans, including T-scores and BMD (measured in g/cm2), were obtained for the second through fourth lumbar vertebrae. T scores are used for diagnosis of osteopenia or osteoporosis. Normal BMD: T score>−1; osteopenia: T score≤−1 but >−2.5; osteoporosis: T score≤−2.5 (8).

Bone Turnover Markers

As bone resorption markers, serum level of the C-Terminal Telopeptides of Type I Collagen (CTX, Roche Diagnostics, Indianapolis, IN) was measured by electrochemiluminescent immunoassay. The serum level of tartrate-resistant acid phosphatase isoform 5b (TRACP-5b, LSBio, Seattle, WA) were measured by enzyme-likned immunosorbent assay. As bone formation markers, serum levels of bone alkaline phosphatase (BALP, Hybritech, Beckman Beckman-Coulter, Brea, CA) and procollagen type I N-terminal peptide (PINP, LSBio, Seattle, WA) were determined by an enzyme-likned immunosorbent assay.

Osteoclast Differentiation

Peripheral blood monocytes (PBMCs) from healthy subjects were collected using Lymphoprep (Stemcell technologies, Vancouver, Canada) gradient centrifugation. Positive selection of monocytes was performed using CD14 MACS microbeads (Miltenyi Biotec, Auburn, CA) according to the protocol supplied by the manufacturer. Purified CD14+ monocytes (1.0×105 cells/well) were left overnight for osteoclast precursors in 96-well plates in αMEM (Thermo Fisher Scientific, St Louis, MO), 10% Hyclone fetal bovine serum (GE Healthcare Life Sciences, Logan, UT), and 1% glutamine (200 mM, Thermo Fisher Scientific, St Louis, MO) with M-CSF (20 ng/mL, PeproTech, Rocky Hill, NJ) and were further cultured for 3 days with M-CSF (20 ng/mL) and RANKL (40 ng/mL, PeproTech, Rocky Hill, NJ).

Osteoclast cells were fixed and stained for TRAP using the Acid Phosphatase Leukocyte diagnostic kit (Sigma Aldrich, Carlsbad, CA) as recommended by the manufacturer. Multinucleated (>3 nuclei), TRAP-positive osteoclasts were counted in triplicate wells.

Flow Cytometry

PBMCs were incubated with saturating concentrations of the monoclonal antibodies at 4° C. for 25 min. Subsequently, red cells were lysed with ACK Lysing Buffer (Gibco Life Technologies, Grand Island, NY), washed twice, resuspended in PBS containing 1% BSA and analyzed on a FACSCanto II (BD Life Sciences, Franklin Lakes, NJ) or FACS Symphony (BD Life Sciences, Franklin Lakes, NJ). For cell sorting, stained cells were fractionated by using a BD Influx Cell Sorter (BD Life Sciences, Franklin Lakes, NJ). The monoclonal antibodies used for the determination of the cell surface phenotype are listed in Table 1.

TABLE 1
Monoclonal antibodies used for the determination
of the cell surface phenotype

| Cluster designation | Identity/function | Clone |
|---|---|---|
| CD1c | Dendritic cells | F10/21A3 |
| CD3 | T cell marker | OKT3 |
| CD4 | Receptor for MHC class II | SK3 |
| CD8 | Receptor for MHC class I | SK1 |
| CD14 | Monocytes | M5E2 |
| CD15 | Granulocytes | W6D3 |
| CD16 | Non classical monocytes | 3G8 |
| CD19 | B cell marker | SJ25C1 |
| CD51/CD61 | Osteoclasts, endothelial cells, | 23C6 |
| | and melanoma cells | |
| CD66b | Granulocytes | G10F5 |
| CD161 | NK cell | DX12 |
| CD235a | Erythrocytes | HI264 |
| C3AR1 | Chemotactic and | hC3aRZ8 |
| | inflammatory factors | |
| CCR2 | Monocyte chemoattractant | K036C2 |
| HLA-DR | MHC II | G46-6 |
| PDL-1 | T cell activation | 29E.2A3 |
| RANK | Osteoclast differentiation and | MIH24 |
| | activation | |

Cytospin Preparation and Staining

For cytologic analysis of cell preparations, 5000 cells were mounted on slides using a Cytospin centrifuge for 5 min at 1000 RPM. Cells were air-dried, then fixed and stained using a Giemsa stain according to manufacturer's instructions. Cytospin preparations were imaged using a standard light microscope using a 40× (Nikon, Melville, NY).

Immunohistochemistry

CD14 positive cells (0.5×10⁶ cells/well) were allowed to adhere on 4 chamber slide well plates with M-CSF overnight and were further cultured with M-CSF and RANKL for 1 day. Cells were fixed in 4% paraformaldehyde for 20 min and treated with 0.2% Triton X for 5 min. Samples were washed three times with PBS, blocked in blocking solution [1% (w/v) BSA (Sigma Aldrich, UK), 5% (v/v) nonspecific goat serum (Millipore Sigma, St. Louis, MO), 5% (v/v) nonspecific donkey serum (Millipore Sigma, St. Louis, MO)] for 20 min at room temperature. Samples were incubated with primary NFATc1 antibody (1:200, Biolegend, San Diego, CA) overnight at 4° C. Next day the samples were washed and incubated with AlexaFluor 488-conjugated anti-mouse IgG as secondary antibody (1:400, Jackson Immunoresearch, West Grove, PA) for 30 min at room temperature, and then counterstained with DAPI (Invitrogen, Carlsbad, CA) Images were captured with a microscope.

RNA-Sequencing

Four biological replicates from two independent donors were used for RNA-sequencing. Total RNA was extracted using RNeasy mini kit (Qiagen, Hilden, Germany) True-seq RNA Library preparation kits (Illumina, Inc., San Diego, CA) were used to purify poly-A+ transcripts and generate libraries with multiplexed barcode adaptors following the manufacturer's instructions. All samples passed quality control analysis on a Bioanalyzer 2100 (Agilent Technologies, Santa Clara, CA). Paired-end reads were obtained on an Illumina HiSeq 2500 in the Weill Cornell Medical College Genomics Resources Core Facility or the Weill Cornell Epigenomics Core Facility. Read quality was assessed with FastQC v0.11.6 and adapters trimmed using Cutadapt v1.15. Reads were then mapped to the human genome (hg38) and reads in exons were counted against Gencode v27 with STAR Aligner v2.5.3a. Differential gene expression analysis was performed in R v3.5.1 using edgeR v3.20.9. Genes with low expression levels (<3 cpm in at least one group) were filtered from all downstream analyses. The Benjamini-Hochberg false discovery rate procedure was used to calculate q-value. Genes with p-value>0.01 and log2 (fold-change)<0.5 were filtered out.

Statistical Analysis

Statistical analysis was performed with Prism ver. 8.0 software (Graphpad Software, San Diego, CA). Numerical data were expressed as both the mean±SD and the median with the interquartile range. When comparing two groups, the unpaired t-test was applied for numerical data. Multiple comparisons were performed by one-way ANOVA followed by Tukey multiple comparison test. Spearman's rank correlation coefficient test was employed to evaluate the correlation of assessed parameters. The level of significance was set at P<0.05.

Example 2—Results

Patient Characteristics

Table 2 shows the demographic and clinical data of the osteoporosis patients, osteopenia patients and normal BMD subjects. In our study, the all menopausal women were divided into three groups based on their T-score at the lumbar vertebrae or total hip sites. The mean age, average age at menopause, serum 25-hydroxyvitamin D, serum calcium and serum phosphorus showed no significant differences among three groups. Body mass index of patients with osteoporosis tended to be lower than those of patients with osteopenia and normal BMD subjects, but there were no statistically significant difference. Naturally, the BMD values and T-scores (both lumbar spine and total hip) in patients with osteoporosis were significantly lower than those in osteopenia patients and normal BMD subjects. Serum level of CTX and BALP in osteoporosis patients tended to be higher than those in osteopenia patients and normal BMD subjects.

TABLE 2
Demographics and clinical data at baseline of the study population

| | Normal bone density | | Osteopenia | | Osteoporosis | | |
|---|---|---|---|---|---|---|---|
| | T-score ≥ −1.0 | | −2.5 < T-score < −1.0 | | T-score ≤ −2.5 | | |
| | n = 9 | | n = 19 | | n = 16 | | P value |
| Age (years) | 66.1 ± 9.5 | | 65.6 ± 6.6 | | 67.5 ± 10.0 | | 0.8977 |
| | 70.0 | [56.0-74.0] | 65 | [59-70] | 69.5 | [61.8-74.3 | |
| Menopause age (years) | 50.0 ± 4.1 | | 49.1 ± 3.5 | | 50.1 ± 3.8 | | 0.7338 |
| | 50 | [47.5-52.0] | 50 | [47.0-51.0] | 50 | [45.8-53.8] | |
| Body mass index (kg/m2) | 24.1 ± 1.7 | | 23.8 ± 3.8 | | 21.6 ± 2.8 | | 0.1143 |
| | 23.9 | [23.7-25.7 | 23.8 | [19.9-27.1] | 21.2 | [19.3-24.2] | |
| Lumbar spine BMD (T-score) | −0.03 ± 0.60 | | −1.55 ± 0.73 | | −2.8 ± 0.8 | | <0.0001 |
| | −0.30 | [−0.50-−0.7] | −1.80 | [−2.1-−0.9] | −2.55 | [−3.15-−2.08] | |
| Lumbar spine BMD (g/cm2) | 1.06 ± 0.10 | | 0.87 ± 0.07 | | 0.77 ± 0.09 | | <0.0001 |
| | 1.04 | [0.97-1.11 | 0.87 | [0.82-0.92] | 0.79 | [0.73-0.84 | |
| Total hip BMD (T-score) | −0.77 ± 0.88 | | −1.57 ± 0.39 | | −2.19 ± 0.47 | | 0.0006 |
| | −1.0 | [−1.5-−0.1] | −1.50 | [−1.73-−1.33] | −2.10 | [−2.63-−1.85] | |
| Total hip BMD (g/cm2) | 0.83 ± 0.12 | | 0.76 ± 0.06 | | 0.66 ± 0.11 | | 0.0070 |
| | 0.79 | [0.75-0.95] | 0.76 | [0.74-0.78] | 0.70 | [0.59-0.74 | |
| 25(OH)D (ng/ml) | 48.3 ± 13.9 | | 37.8 ± 9.8 | | 43.4 ± 12.1 | | 0.2656 |
| | 50.0 | [35.8-57.0 | 38.3 | [32.1-48.0] | 47.2 | [30.8-58.3] | |
| Calcium (mg/dL) | 9.3 ± 0.8 | | 9.6 ± 0.3 | | 9.5 ± 0.4 | | 0.7040 |
| | 9.3 | [8.6-10.1] | 9.6 | [9.4-10.0] | 9.7 | [9.4-9.9] | |
| Phosphorus (mg/dl) | 3.5 ± 0.8 | | 3.7 ± .5 | | 3.7 ± 0.5 | | 0.9235 |
| | 3.7 | [2.7-4.2 | 3.8 | [3.5-4.1] | 3.7 | [3.2-3.9] | |
| CTX (pg/ml) | 264.5 ± 177.0 | | 374.5 ± 174.4 | | 443.0 ± 201.3 | | 0.1846 |
| | 253.5 | [99.5-440.5] | 378.5 | [253.5-427.3] | 448.0 | [264.8-609.0] | |
| BALP (U/L) | 11.1 ± 4.1 | | 12.8 ± 6.8 | | 15.1 ± 6.5 | | 0.9184 |
| | 13.50 | [6.4-13.5] | 11.5 | [8.3-14.9] | 12.3 | [8.6-21.2] | |

Data are mean ± SD and median [25th to 75th percentile range],
BMD: bone mineral density,
25(OH)D: 25-hydroxyvitamin D,
CTX: C-terminal type 1 collagen telopeptide;
BAP: Bone Specific Alkaline Phosphatase

RANK Positive Cells Identification in Monocytes

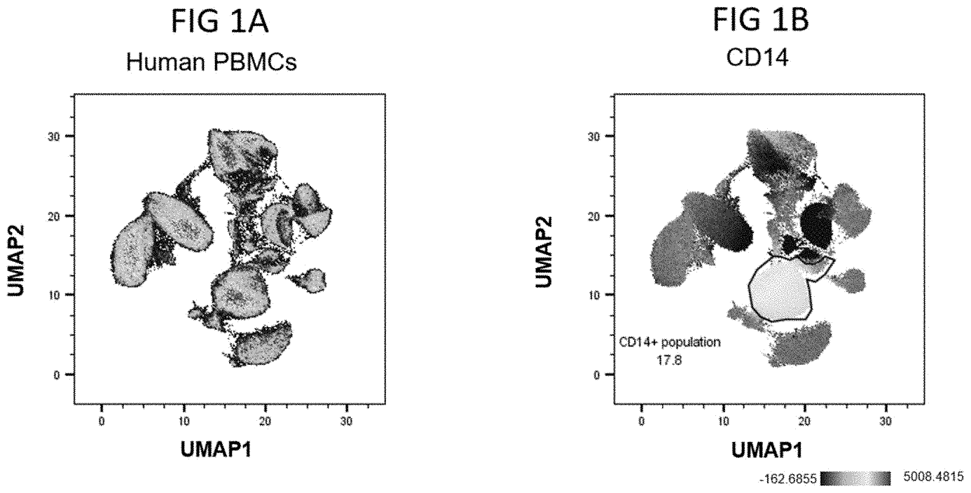

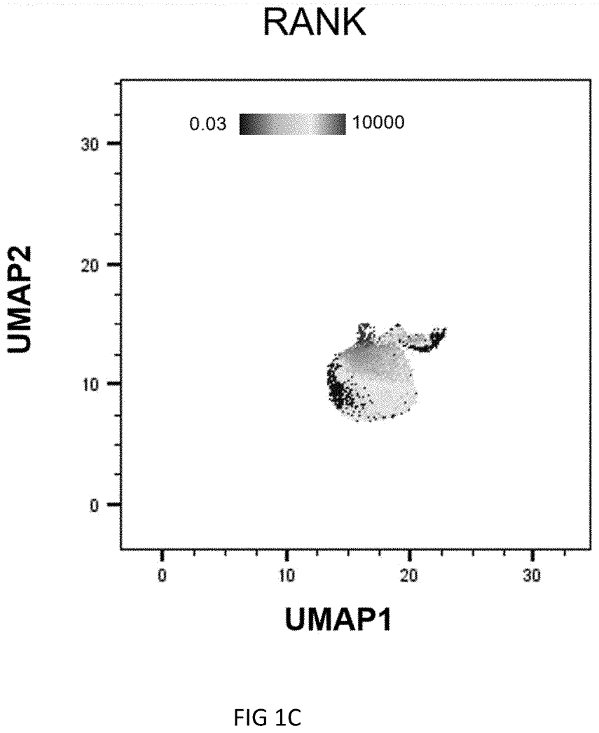

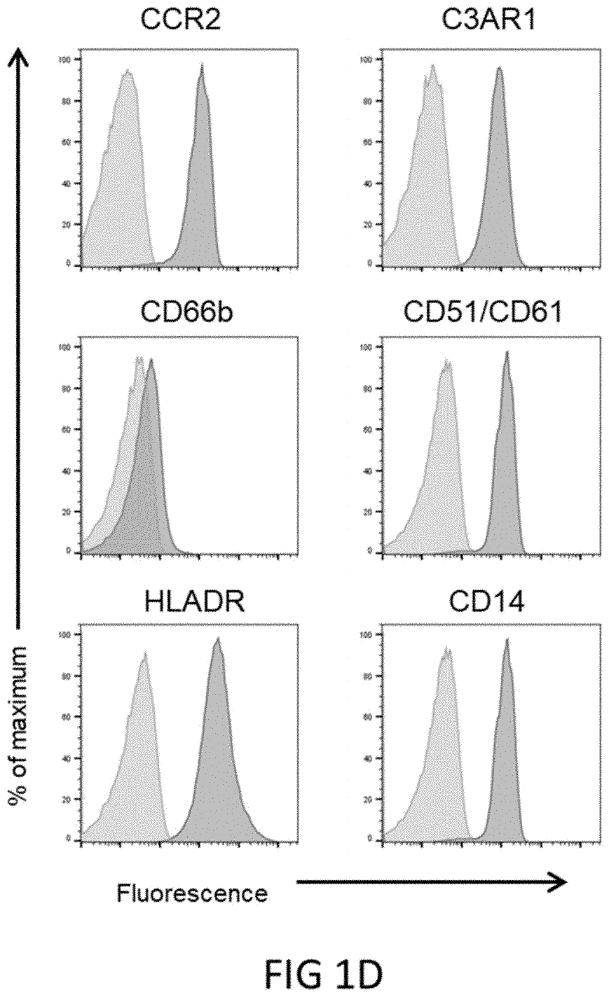

To visualize RANK subsets in healthy subjects, two dimensional UMAP plots were generated (FIG. 1A). Starting with the PBMCs population and using the markers CD14, a distinct CD14+ monocyte cell cluster could readily be identified (FIG. 1B). To zoom in on the CD14+ cell population only, a new UMAP was generated. In this third UMAP, most RANK+ cells could be identified in CD14+ cell abundant population (FIG. 1C).

The expression of CCR2, C3AR1, CD51/61, CD14, CD66b and HLA-DR on the CD14+/RANK+ cells were investigated for PBMCs in 3 healthy subjects. The expression of CCR2, C3AR1, CD14 and HLA-DR was clearly increased, and the expression of CD51/CD61 and CD66b was quite elevated (FIG. 1D).

Osteoclast Enhancing Cells (OECs) Enhanced Osteoclastogenesis

To examine whether there is an imbalance of subpopulations of monocytes, and whether RANK expression is disturbed in different subpopulations of monocytes in healthy subjects, we used CD14, CD66b and RANK to separate monocytes into different subsets. FIG. 2A shows a representative flow plot of human PBMCs.

Starting from PBMCs, here monocyte subsets were identified on the basis of forward scatter and side scatter characteristics. Following exclusion of debris and cellular aggregates, a live/dead discrimination was performed. They were positively gated for CD14, and then we found two subpopulations which were distinguished by their surface expression of RANK. RANK high/CD66b negative cells on CD14+ cells are named as osteoclast-enhancing cells (OECs) and RANK negative/CD66b negative on CD14+ cells are named as monocytes (MOs) (FIG. 2A, bottom plot).

We investigated the distribution of fluorescence intensity of CCR2, C3AR1, CD51/CD61 and HLA-DR expression levels in OECs. Representative results from one of these three healthy subjects with normal BMD are presented in FIG. 2B. PBMCs from 3 healthy subjects demonstrated increased cell surface expression of CCR2, C3AR1, CD51/CD61 and HLA-DR in OECs.

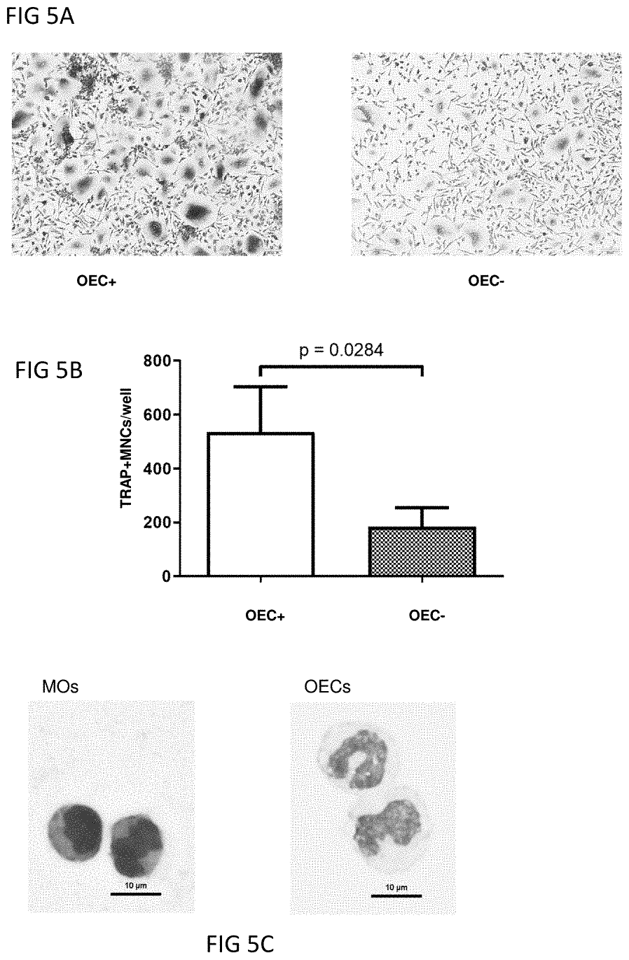

To identify the monocyte subset that differentiates into osteoclasts, cell sorting analysis was performed and then we examined osteoclast formation from OECs and MOs in healthy subjects with normal BMD. PBMCs from healthy subjects with normal BMD were stained with RANK antibody, CD14 antibody, CD235a antibody and CD66b antibody, and were fractionated by a cell sorter. Thus, two populations, OECs and MOs were separated [FIG. 2A (bottom right panel)]. Each sorted cell was cultured in the presence of M-CSF for 1 day and was further cultured for 3 days with M-CSF and RANKL. Culture with M-CSF and RANKL induced a significant number of TRAP-positive MNC from the OECs, but MOs differentiated into TRAP-positive MNC less than that of OECs (FIG. 5A). FIG. 5B shows the percentage of TRAP-positive multinuclear cells (MNCs: more than three nuclei) per control.

We then examined the appearances of these two cell populations. OECs and MOs showed different morphologies. OECs were larger granular mononuclear cells, while MOs are smaller mononuclear cells (FIG. 5C).

RANKL Enhanced Expression of NFATc1 in Osteoporosis Patient

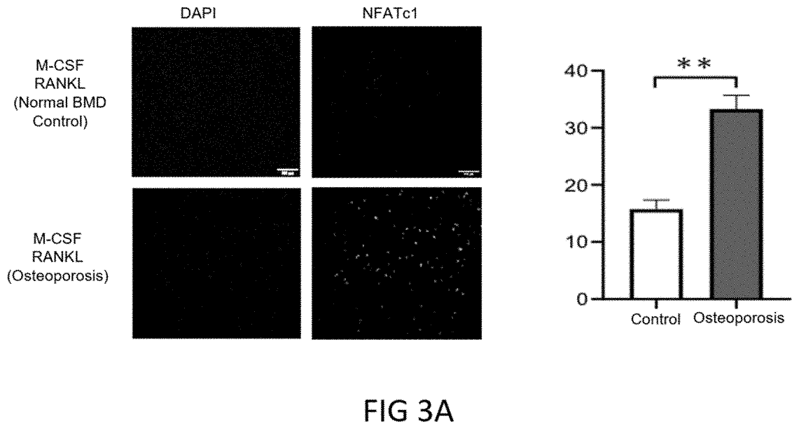

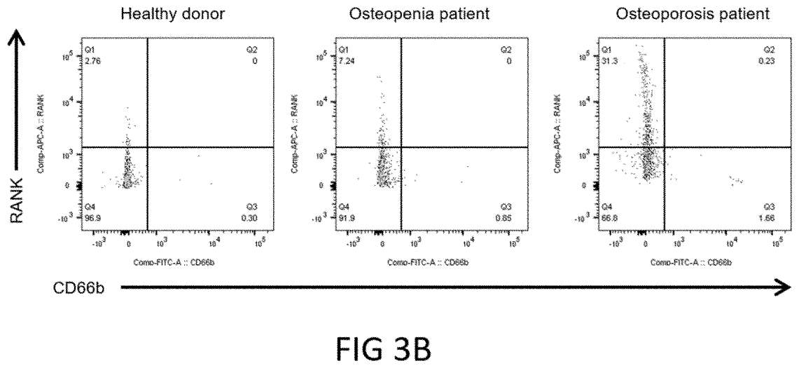

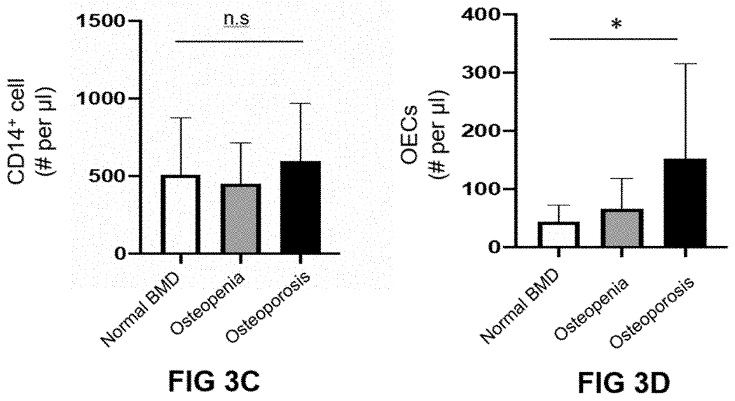

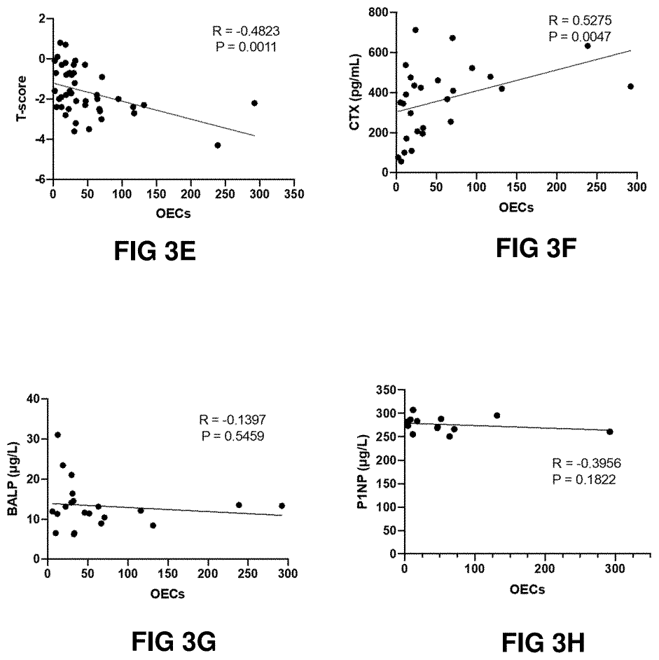

NFATc1 plays a critical role in osteoclast formation and function (9). We examined the expression of NFATc1 on CD14+ monocytes by immunofluorescent staining. Neither unstimulated nor M-CSF-stimulated monocyte subsets expressed NFATc1 (data not shown). As shown in FIG. 3A (left panel), after 24 hours of treatment with M-CSF+ RANKL, NFATc1-positive mononuclear cells were abundantly observed in the cultures of osteoporosis patients, but few in the cultures of healthy subject with normal BMD. FIG. 3A (right panel) showed the percentage of NFATc1 positive cells per total cells in osteoporosis patients was significantly higher than that in healthy subject with normal BMD.

OECs Increase in Patients with Osteoporosis

We used RANK antibody, CD14 antibody, CD235a antibody and CD66b antibody in flow cytometric analysis to detect OECs and MOs. The representative percentage of OECs in patients with normal BMD, osteopenia and osteoporosis are shown in FIG. 3B. Our results showed that the percentage of OECs was increased in osteoporosis patients as compared to normal BMD subjects and osteopenia, but there was no difference between normal BMD subjects and osteopenia.

We further analyzed the difference in the absolute number of CD14+ cells among three groups. There were no statistically significant differences in the absolute number of CD14+ cells among the three groups (FIG. 3C).

The absolute number of OECs among the three groups are shown in FIG. 3D. The number of OECs was significantly higher in osteoporosis patients than those in normal BMD subjects and osteopenia (p=0.005 and p=0.008, respectively), but there was no difference between normal BMD subjects and osteopenia (p=0.770).

Correlation Between OECs and BMD or Bone Turnover Markers

We further analyzed whether there were correlations between the number of OECs with spine BMD or BTMs. Our results showed that the number of OECs were significantly negatively correlated with lumbar spine BMD values and T-score in all subjects (r=−0.466; P=0.002, r=−0.412; P=0.006, respectively) (FIG. 3E). As shown in FIG. 3F, the serum level of CTX, a bone resorption marker, was significantly positively correlated with number of OECs (r=0.452; P=0.018), whereas serum levels of the BALP, a bone formation marker, did not correlate with the number of OECs (r=0.3499; P=0.131) (FIG. 3G).

RNA-Seq Analysis in OECs and MOs

In total, 10000 differentially expressed RNAs were identified between OECs and MOs from osteoporosis patients and normal BMD subjects, with FDR<0.01 and 1.5 fold changes. The expression profiles of abnormally expressed RNAs were demonstrated by volcano plot in FIG. 4A. Significantly regulated genes in MOs compared to OECs were 796, whereas the genes that ware highly regulated in OECs was 130.

FIG. 4B shows that enriched canonical pathways of the differentially expressed genes using Ingenuity Pathway Analysis (IPA). The significance of canonical pathways was determined by IPA's default threshold [−log (p-value)>1.3].

Heatmap showing relative expression (z-score) of 926 genes differentially expressed with FDR<0.01 in OECs versus MOs from 2 replicates (FIG. 4C). One sample contains cumulative data from 6 donors and the other sample has cumulative data from 5 donors.

Example 3

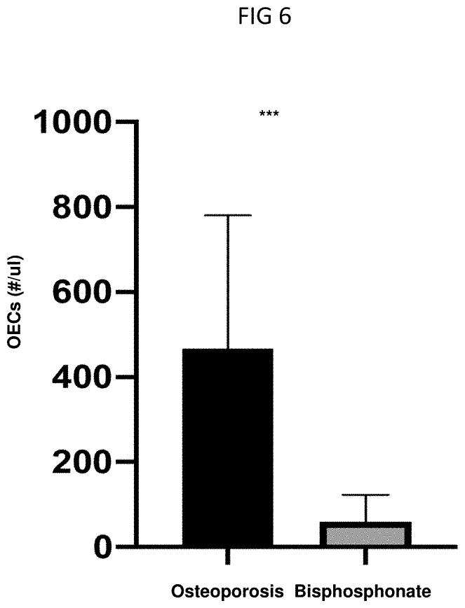

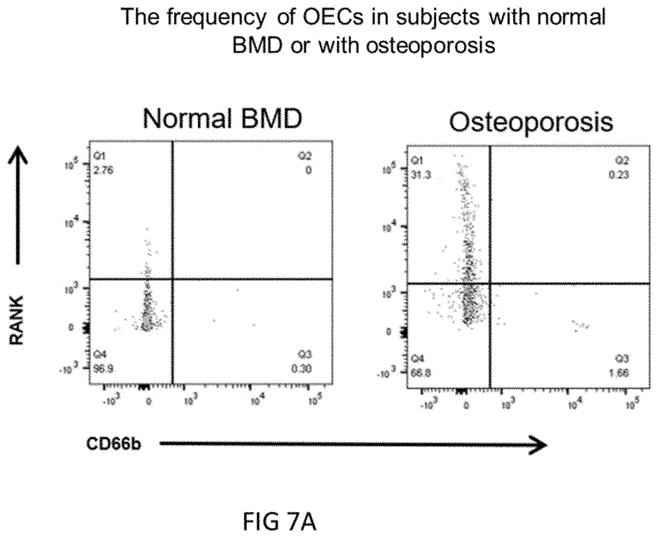

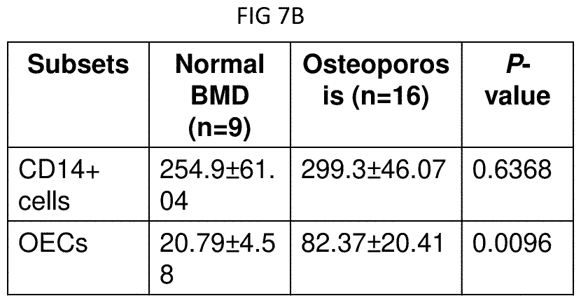

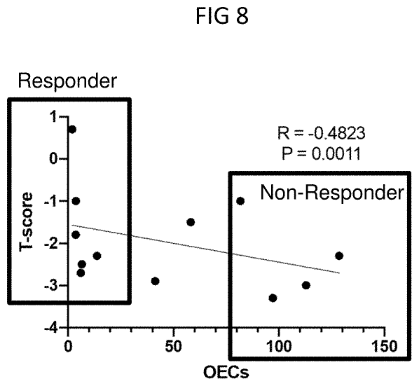

FIG. 6 is a plot showing the number of OECs in patients with osteoporosis with or without bisphosphonate treatment. Bisphosphonate (n=11) and No treatment group (n=16). FIG. 6 demonstrates that bisphosphonates decrease circulating OECs in osteoporosis patients. The number of circulating OECs in subjects with normal BMD or with osteoporosis is shown in FIGS. 7A-7B. The osteoporosis group (n=16) is the same as in FIG. 6.

Example 4

Bone mineral density (BMD) was measured for the bisphosphonate treatment group. FIG. 8 shows BMD (shown as T-score) plotted versus number circulating OECs in osteoporosis patients treated with bisphosphonate as in Example 3. It can be seen that the “Responders”, i.e., those with higher BMD scores have lower circulating OECs as compared to “Non-responders”.

All documents cited in this specification are incorporated herein by reference in their entireties, as is U.S. Provisional Patent Application No. 63/069,477, filed Aug. 24, 2020. While the invention has been described with reference to particular embodiments, it will be appreciated that modifications can be made without departing from the spirit of the invention. Such modifications are intended to fall within the scope of the appended claims.

References
1. National Osteoporosis Foundation. Fast Facts. http://www.nof.org/node/40. Accessed on Jul. 25, 2020.
2. J. J. Roche, R. T. Wenn, O. Sahota, C. G. Moran, Effect of comorbidities and postoperative complications on mortality after hip fracture in elderly people: prospective observational cohort study. BMJ 331, 1374 (2005).
3. G. M. Blake, I. Fogelman, The role of DXA bone density scans in the diagnosis and treatment of osteoporosis. Postgrad Med J 83, 509-517 (2007).
4. C. H. Chesnut, 3rd, McClung, M. R., Ensrud, K. E., Bell, N. H., Genant, H. K., Harris, S. T., Singer, F. R., Stock, J. L., Yood, R. A., and Delmas, P. D, Alendronate treatment of the postmenopausal osteoporotic woman effect of multiple dosages on bone mass and bone remodeling. Am J Med 99, 144-152 (1995).
5. R. Civitelli, R. Armamento-Villareal, N. Napoli, Bone turnover markers: understanding their value in clinical trials and clinical practice. Osteoporos Int 20, 843-851 (2009).
6. T. T. Hlaing, J. E. Compston, Biochemical markers of bone turnover—uses and limitations. Ann Clin Biochem 51, 189-202 (2014).
7. K. J. Bell et al., The potential value of monitoring bone turnover markers among women on alendronate. J Bone Miner Res 27, 195-201 (2012).
8. G. M. Blake, K. M. Knapp, I. Fogelman, Dual X-ray absorptiometry: clinical evaluation of a new cone-beam system. Calcif Tissue Int 76, 113-120 (2005).
9. K. H. Park-Min, Mechanisms involved in normal and pathological osteoclastogenesis. Cell Mol Life Sci 75, 2519-2528 (2018).
10. U.S. Pat. No. 7,807,389 B2.
11. Lianping Xing and Edward M. Schwarz, Circulating Osteoclast Precursors: A Mechanism and a Marker of Erosive Arthritis, 2005, 1:1(21-28).
12. Harry C. Blair and Jill L. Carrington, Bone Cell Precursors and the Pathophysiology of Bone Loss, April 2006, 1068(1):244-249.
13. Perkins SL et al, Age-related bone loss in mice is associated with an increased osteoclast progenitor pool, Bone, Volume 15, Issue 1, January-February 1994, Pages 65-72.
14. Charles JF et al, Inflammatory arthritis increases mouse osteoclast precursors with myeloid suppressor function, JCI, December 2012, 122(12):4592-4605.
15. Gossiel, F., Hoyle, C., McCloskey, E.V. et al. (4 more authors) (2016) The effect of bisphosphonate treatment on osteoclast precursor cells in postmenopausal osteoporosis: The TRIO study. Bone, 92. pp. 94-99. ISSN 8756-3282.
16. D. L. Lacey et al., Bench to bedside: elucidation of the OPG-RANK-RANKL pathway and the development of denosumab. Nat Rev Drug Discov 11, 401-419(2012).
17. S. R. Cummings et al., Denosumab for prevention of fractures in postmenopausal women with osteoporosis. N Engl J Med 361, 756-765 (2009).
18. R. Eastell et al., Postmenopausal osteoporosis. Nat Rev Dis Primers 2, 16069 (2016).

Listing of claims:

1. A method of diagnosing and treating a skeletal disease in a subject, the method comprising:
(i) identifying the presence of osteoclast-enhancing cells (OECs) in the subject;
(ii) quantifying the number of circulating OECs in a sample from the subject; and
(iii) diagnosing a skeletal disease in the subject when an increase in OECs is detected as compared to a control.

2. The method of claim 1, further comprising treating the subject for the skeletal disease.

3. The method of claim 1, wherein the skeletal disease is osteoporosis.

4. The method of claim 1, wherein the skeletal disease is osteopenia.

5. The method of claim 1, wherein OECs are detected by identification of cell surface markers.

6. The method of claim 5, wherein the OECs have the phenotype CD14+/RANK+/CD66−.

7. The method of claim 1, wherein the OECs have the phenotype CD14+RANK+CD66b−CCR2+C3AR+CD51/61+HLADR+.

8. The method of claim 1, wherein the subject is treated for osteoporosis using antiresorptive therapy.

9. (canceled).

10. A method of assessing the efficacy of a treatment for a skeletal disease, the method comprising:
(i) identifying the presence of osteoclast-enhancing cells (OECs) in a subject that has received treatment for the skeletal disease; and
(ii) quantifying the number of circulating OECs in a sample from the subject;
wherein a decrease in the number of circulating OECs as compared to a control indicates the treatment is at least partially efficacious.

11. The method according to claim 10, wherein the treatment is a bisphosphonate.

12. The method of claim 10, wherein the control is an OEC level obtained from the subject at an earlier time point.

13. A method of diagnosing an increased risk of a surgical complication in a subject prior to surgery, the method comprising:
(i) identifying the presence of osteoclast-enhancing cells (OECs) in the subject;
(ii) quantifying the number of circulating OECs in a sample from the subject;
(iii) diagnosing an increased risk of a surgical complication in the subject when an increase in OECs is detected as compared to a control.

14. (canceled).

15. The method of claim 10, wherein the treatment is bisphosphonate treatment, and wherein a level of 80 cells/uL or less indicates that the subject is responding to bisphosphonate treatment.
